(19) United States
(12) Patent Application Publication
(10) Pub. No.: US 2002/0147176 A1
(43) Pub. Date: Oct. 10, 2002

(54) Thermoplastic and water-soluble cellulose ether esters

(76) Inventors: Felix Ecker, Leverkusen (DE); Venkata-Rangarao Kanikanti, Leverkusen (DE); Klaus Elgeti, Bergisch Gladbach (DE); Bernd Tieke, Bruhl (DE)

Correspondence Address: Jeffrey M. Greenman, Vice President, Patents and Licensing, Bayer Corporation, 400 Morgan Lane, West Haven, CT, 06516, US

(21) Appl. No.: 10/005,566

(22) Filed: Dec. 5, 2001

(30) Foreign Application Priority Data
Dec. 5, 2000 (DE) 10060470.6

(51) Int. Cl. A61K 31/717; C08B 3/00

(52) U.S. Cl. 514/57; 536/63

(57) ABSTRACT

The present invention relates to thermoplastic, water-soluble cellulose derivatives of the general formula

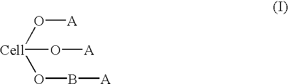

in which

Cell is the substituted residue of a hydroxyl group on the cellulose chain,

A is hydrogen or a hydroxycarboxylic acid residue,

B is an ether residue (—E—O)ₙ,

in which

n is 1 to 4, the molar degree of substitution by hydroxycarboxylic acid being between 0 and 1 and the molar degree of substitution of the ether being greater than or equal to 3,

E represents C₁-C₆-alkyl.

The present invention relates to novel thermoplastic and water-soluble cellulose ether esters of lactic acid and of hydroxyacetic acid of the general formula (I):

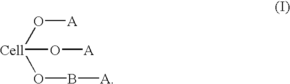

Accurate control of the release of active ingredients from preparations is of great pharmaceutical importance. Besides rapid-release preparations, which ensure a rapid increase in the level of active ingredient in the circulation for an acute pathological state, those with modified release of active ingredients, in particular slow-release preparations, are frequently employed.

The principle of melt extrusion has already been known for a long time (Beckmann 1964). Application of melt extrusion technology to the development of novel pharmaceutical forms with modified release of active ingredient is, however, a relatively new process. It entails active ingredient and polymer being conveyed either simultaneously, without previous mixing, or as mixture, after previous mixing, in an extruder which has been heated so that the mixture is extrudable and the active ingredient is not degraded. In contrast to conventional coprecipitate methods, the use of solvents is unnecessary in this case: this is particularly important because, besides economic aspects, the use of solvents gives rise to special technical problems such as prevention of explosion in the premises and equipment.

Despite the apparent multiplicity of alternatives, the skilled person developing formulations is often confronted by great difficulties because the desired active ingredient can be formulated only inadequately or not at all with the systems available.

The cellulose-based thermoplastic and water-soluble polymer mainly used at present is hydroxypropylcellulose. Thus EP-A-806 433 describes thermoplastic and water-insoluble cellulose ether 2-hydroxycarboxylic acid esters and mixed esters. EP-A-626 392 describes thermoplastic and water-insoluble cellulose ether hydroxycarboxylic acid esters.

The insolubility in water of the described polymers in the described systems is disadvantageous in pharmaceutical use because the system does not completely dissolve in the body, and the active ingredient thus possibly cannot be released in adequate quantity.

One object of the present invention was therefore to provide alternative thermoplastic, water-soluble cellulose ether esters and a process for their preparation, it being possible to adjust various property profiles.

The present invention therefore relates to thermoplastic, water-soluble cellulose derivatives of the general formula

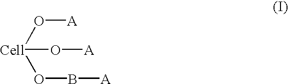

in which

Cell is the substituted residue of a hydroxyl group on the cellulose chain,

A is hydrogen or a hydroxycarboxylic acid residue,

B is an ether residue (—E—O)ₙ,

in which

n is 1 to 4, the molar degree of substitution by hydroxycarboxylic acid being between 0 and 1 and the molar degree of substitution of the ether being greater than or equal to 3,

E represents C₁-C₆-alkyl.

The compounds of the present invention are suitable for producing pharmaceutical preparations for the treatment of diseases. Pharmaceutical preparation means any one suitable form making it possible to administer an active ingredient. These include, for example, tablets, film-coated tablets, sugar-coated tablets, granules, powders, suspensions, emulsions, solutions, gels, ointments. The compounds according to the invention may serve for this purpose as filler, gel former, coating material, thickener, capsule shell material or embedding matrix. They are used in particular as embedding matrix for producing tablets and granules.

Preferred hydroxycarboxylic acids are α-hydroxycarboxylic acids, in particular lactic acid and hydroxyacetic acid. The molar degree of substitution (MS) of the cellulose derivative by hydroxycarboxylic acid is between 0 and 1, that is to say greater than 0 and less than 1. Molar degree of substitution refers in this connection to the average number of moles of hydroxycarboxylic acid reacted per anhydroglucose unit of the cellulose.

Suitable ethers (—E—O)ₙ, are in principle all customary linear or branched hydrocarbon structures, in particular with 1 to 6 carbon atoms. Propyl is particularly preferred. The molar degree of substitution (MS) by ether should be greater than or equal to 3, in particular 3 to 4.5, especially 3.5 to 4, the molar degree of substitution referring to the average number of moles of alkene oxide (for example propylene oxide) reacted per anhydroglucose unit of the cellulose.

The present invention additionally relates to a process for preparing such water-soluble thermoplastic cellulose ether esters by transesterification of hydroxy-propylcellulose with esters of the appropriate hydroxycarboxylic acids, in particular dilactide or 1,4-dioxane-2,5-dione. The reaction is carried out heterogeneously as suspension in dioxane without catalyst.

The cellulose ether esters according to the invention can be converted into pharmaceutical preparations with the aid of conventional extrusion processes. These include, for example, melt extrusion with screw or ram extruder, in particular melt extrusion with single- or twin-screw extruder.

It is possible in this connection for polymer and active ingredient to be mixed either before or during the extrusion. Mixing beforehand is preferred.

### SYNTHESIS OF HYDROXYPROPYLCELLULOSE LACTATES

HPC lactates were prepared by transesterification of hydroxypropylcellulose with dilactide, the cyclic dimeric ester of lactic acid. The reaction is carried out heterogeneously as suspension in dioxane without catalyst.

The quantities of dilactide and dioxane employed are stated as multiple of the number of moles of anhydroglucose units in the initial hydroxypropylcellulose and are referred to hereinafter as L/G molar ratio. The HPC lactates are characterized in this case by indicating in parentheses first the degree of lactate substitution and then the batch number, for example HPC lactate (0.79; 04).

0.3 mol of hydroxypropylcellulose (based on anhydroglucose units) was introduced into the 21 reactor, and 0.09 mol into the 51 reactor, and suspended respectively in 13.4 mol and 40.1 mol of dioxane. This corresponds to a dioxane/glucoside molar ratio of 44.6. The individual batches with the quantities of dilactide employed in each case and the MS_{lactate} resulting therefrom are described in Table 1.

Hydroxypropylcellulose (for example Klucel®) itself and its preparation are known, for example from K. Engelskirchen in Houben-Weyl, Methoden der organischen Chemie [Methods of organic chemistry], Volume E20, additional and supplementary volume to the 4th edition, Georg Thieme Verlag, Stuttgart, New York, 1987, or Hercules Inc., Klucel Hydroxypropylcellulose—Physical and Chemical Properties, product documentation, September 1997.

The degrees of substitution (MS_{HP}) by ether of the HPC types used for synthesizing the cellulose ether esters are:

| | |
|---|---|
| Klucel HXF | MS_{HP} = 3.9 |
| T 588 | MS_{HP} = 4.0 |
| T 587 | MS_{HP} = 3.85 |
| T 595 | MS_{HP} = 3.64 |

TABLE 1
Batches for synthesizing HPC lactates

| | | | L/G | | Yield | |
|---|---|---|---|---|---|---|
| Batch | Initial | Sub- | molar | | MS_{lactate}/ | Reactor |
| number | HPC | stituent | ratio | MS_{lactate} | L/G | [liters] |
| 01 | T588 | L-Lactide | 1.8 | 0.87 | 48% | 3 |
| 02 | T588 | L-Lactide | 1.0 | 0.54 | 54% | 3 |
| 03 | T588 | L-Lactide | 2.6 | 1.03 | 40% | 3 |
| 04 | T588 | L-Lactide | 1.8 | 0.79 | 44% | 5 |
| 05 | T588 | L-Lactide | 1.2 | 0.60 | 50% | 5 |
| 06 | T588 | L-Lactide | 0.6 | 0.40 | 67% | 5 |
| 11 | T587 | L-Lactide | 1.8 | 0.73 | 41% | 5 |
| 12 | T587 | D-Lactide | 1.8 | 0.71 | 39% | 5 |
| 21 | T595 | L-Lactide | 1.8 | 0.76 | 42% | 5 |
| 22 | T595 | L-Lactide | 1.2 | 0.63 | 52% | 5 |
| 23 | T595 | L-Lactide | 0.6 | 0.38 | 63% | 5 |
| 24 | T595 | D-Lactide | 1.8 | 0.80 | 44% | 5 |
| 31 | Klucel HXF | L-Lactide | 0.6 | 0.32 | 53% | 5 |
| 32 | Klucel HXF | L-Lactide | 1.2 | 0.24 | 20% | 5 |
| 33 | Klucel HXF | L-Lactide | 1.8 | 0.29 | 16% | 5 |
| 34 | Klucel HXF | L-Lactide | 0.6 | 0.17 | 28% | 5 |
| 35 | Klucel HXF | L-Lactide | 1.2 | 0.26 | 22% | 5 |
| 36 | Klucel HXF | L-Lactide | 1.8 | 0.33 | 18% | 5 |

The precursors were weighed and introduced into the reactor. After closing, flushing with nitrogen was carried out. This was done by alternately evacuating the reactor and filling it with 5 bar of nitrogen three times. The reactor was finally evacuated again and the pressure was adjusted to 1 bar with nitrogen.

The speed of the anchor stirrer was 50 rpm.

The reaction was started by heating the batch to 130° C. in 60 min. This temperature was kept constant for 5 hours. After the reactor had cooled back to room temperature, the product, which was in the form of a highly viscous gel, was removed from the reactor. The polymer was obtained by precipitation with 51 of hexane, dried at 55° C. and purified by washing twice with hot water. It was finally ground in a Fritsch cutting mill.

The products which are formed are soluble and have flocculation points between 35° C. (batch number 3) and 41° C. (batch number 6).

The product was characterized by ¹³C-NMR spectroscopy of the solid. The spectra of the initial HPC (T595) and of the dilactide and of the HPC lactate (0.76; 21) are described below.

¹³C—NMR spectrum of the solid hydroxypropylcellulose T595:

| | |
|---|---|
| Anhydroglucose: | δ 67.26 ppm (C₆); δ 75.28 ppm (C₂, C₃, C₅); |
| | δ 83.43 ppm (C₄); δ 103.31 ppm (C₁) |
| Hydroxypropyl side chains: | δ 18.21 ppm (R—CH₂—CH₂OR′—CH₃); |
| | δ 20.46 ppm (R—CH₂—CH₂OH—CH₃) |

¹³C—NMR spectrum of the solid L,L-dilactide

| | |
|---|---|
| δ 14.3-16.1 ppm | (—CH₃) |
| δ 73.0 and 74.0 ppm | (—O—CH(CH₃)—) |
| δ 168.8-172.5 ppm | (—O—CO—) |

δ 82.7 ppm (1st spinning side bands of the (—O—CO—) group [92])

The splitting of the peak for the (—O—CO—) group (168.8 ppm; 169.5 ppm; 170.2 ppm; 172.5 ppm) can be explained by the existence of positional isomers and oligomers.

¹³C—NMR spectrum of the solid HPC lactate (0.76; 21):

| | |
|---|---|
| Anhydroglucose: | δ 67.64 ppm (C₆); δ 74.99 ppm (C₂, C₃, C₅); |
| | δ 83.09 ppm (C₄); δ 103.74 ppm (C₁) |
| Hydroxypropyl side chains: | δ 18.17 ppm (R—CH₂—CH₂OR′—CH₃); |
| | δ 20.39 ppm (R—CH₂—CH₂OH—CH₃) |
| Lactate: | δ 170.93 and 175.29 ppm (—O—CO—) |

### SYNTHESIS OF THE HYDROXYPROPYLCELLULOSE GLYCOLIDES

The HPC glycolides were prepared by transesterification of hydroxypropylcellulose with 1,4-dioxane-2,5-dione, the cyclic dimeric ester of hydroxyacetic acid, which is also referred to hereinafter as glycolide for short. The reaction is carried out heterogeneously as suspension in dioxane without catalyst.

The quantities of glycolide and dioxane employed are stated as multiple of the number of moles of anhydroglucose units in the initial hydroxypropylcellulose and are referred to hereinafter as G/G molar ratio. The glycolides are characterized in this case by indicating in parentheses first the degree of substitution and then the batch number, for example HPC glycolide (0.53; 23H).

0.3 mol of hydroxypropylcellulose (based on anhydroglucose units) was introduced into the 21 reactor, and 0.09 mol into the 51 reactor, and suspended respectively in 13.4 mol and 40.1 mol of dioxane. This corresponds to a dioxane/glucoside molar ratio of 44.6. The individual batches with the quantities of glycolide employed in each case and the MS_{glycolide} resulting therefrom are described in Table 2.

TABLE 2
Batches for synthesizing HPC glycolides

| | | Re- | | | | |
|---|---|---|---|---|---|---|
| | | action | | | | |
| | | temp. | G/G | | Yield | |
| Batch | Initial | [° C.] | molar | | MS_{glycolide} | Reactor |
| number | HPC | time [h] | ratio | MS_{glycolide} | G/G | [liters] |
| 22* | T595 | 80/20 | 3 | 2.65 | 88.3% | 5 |
| 31 | Klucel HXF | 80/20 | 2 | 0.45 | 22.5% | 3 |
| 32 | Klucel HXF | 80/20 | 3 | 0.7 | 23.3% | 3 |
| 35 | Klucel HXF | 80/20 | 2 | 0.61 | 30.5% | 5 |
| 21H* | T595 | 130/5 | 2 | 1.80 | 90.0% | 5 |
| 22H | T595 | 130/5 | 1 | 0.95 | 95.0% | 5 |
| 23H | T595 | 130/5 | 0.5 | 0.53 | 106% | 5 |
| 31H* | Klucel HXF | 130/5 | 2 | 1.17 | 58.5% | 5 |
| 34H | Klucel HXF | 130/5 | 1 | 0.72 | 72.0% | 5 |
| 35H | Klucel HXF | 130/5 | 0.5 | 0.42 | 84.0% | 5 |

*comparative examples

The precursors were weighed and introduced into the reactor. After closing, flushing with nitrogen was carried out. This was done by alternately evacuating the reactor and filling it with 5 bar of nitrogen three times. The reactor was finally evacuated again and the pressure was adjusted to 1 bar with nitrogen.

The speed of the anchor stirrer was 50 rpm.

The reaction was started by heating the batch to the reaction temperature in 60 min. This temperature was kept constant throughout the reaction time. The following reaction conditions were investigated:

| | | |
|---|---|---|
| Reaction temperature 80° C.; | reaction time 20 h; | atmospheric |
| | | pressure |
| Reaction temperature 130° C.; | reaction time 24 h; | elevated pressure |
| Reaction temperature 130° C.; | reaction time 5 h; | elevated pressure |

After the reactor had cooled back to room temperature, the product, which was in the form of a highly viscous gel, was removed from the reactor. The polymer was obtained by precipitation with 51 of hexane, dried at 55° C. and purified by washing twice with hot water. It was finally ground in a Fritsch cutting mill. The product was characterized ¹³C-NMR spectroscopy of the solid.

The products of batches 31, 32, 35, 22H, 23H, 24H and 35H are soluble and have flocculation points between 36.8° C. (batch number 34H) and 42.0° C. (batch number 23H). Comparative examples 22, 21H and 31H are insoluble.

¹³C—NMR spectrum of the solid hydroxypropylcellulose Klucel HXF:

| | |
|---|---|
| Anhydroglucose: | δ 66.87 ppm (C₆); δ 75.24 ppm (C₂, C₃, C₅); |
| | δ 83.39 ppm (C₄); δ 102.38 ppm (C₁) |
| Hydroxypropyl side chains: | δ 18.40 ppm (R—CH₂—CH₂OR′—CH₃); |
| | δ 20.45 ppm (R—CH₂—CH₂OH—CH₃) |

¹³C—NMR spectrum of the solid 1,4-dioxane-2,5-dione:

| | |
|---|---|
| δ 60.51 ppm | (—O—CH₂—) |
| δ 168.02 ppm | (—O—CO—) |

δ 81.89 and 87.55 ppm (1st spinning side bands of the (—O—CO—)
group)

The splitting of the peak of the (—O—CO—) group (168.02 ppm; 173.54 ppm; 176.55 ppm) can be explained by the existence of positional isomers and oligomers.

¹³C—NMR spectrum of the solid HPC glycolide (1.83; 33H):

| | |
|---|---|
| Anhydroglucose: | δ 67.06 ppm (C₆); δ 75.42 ppm (C₂, C₃, C₅); |
| | δ 83.27 ppm (C₄); δ 102.84 ppm (C₁) |
| Hydroxypropyl side chains: | δ 18.20 ppm (R—CH₂—CH₂OR′—CH₃); |
| | δ 20.26 ppm (R—CH₂—CH₂OH—CH₃) |
| Glycolide: | δ 168.23 and 172.58 ppm (—O—CO—) |

### DETERMINATION OF THE FLOCCULATION POINT

To find the flocculation point, the polymer was put in a concentration of 0.5% by weight into demineralized water and shaken at room temperature overnight. If the solution was not clear under the conditions, the polymer was designated as insoluble in water and the flocculation point could not be determined. This solution was heated on a magnetic stirrer with hot plate and the temperature of the solution was measured with a thermometer. The flocculation point was defined as the temperature at which a first turbidity of the solution was observable.

### PRODUCTION OF THE EXTRUDATES

The components are mixed in the desired ratio which, in the present experiments, is the ratio 70% by weight polymer and 30% by weight active ingredient. They are then put into an extruder such as a ram extruder, for example a capillary rheometer, and if necessary heated to the required extrusion temperature, in the present case for 15 min. This depends primarily on the active ingredient used. The product was extruded as strand and pelleted with a rotating knife after cooling. In the experiments, the strand was pelleted through a capillary with a diameter of 1 mm.

### RELEASE CHARACTERISTICS OF THE EXTRUDATES WITH NIFEDIPINE AS ACTIVE INGREDIENT

The releases as a % of the single dose of 30 mg of active ingredient were measured by the EP/DAB paddle method with a stirrer speed of 150 rpm. The release medium was a buffer of pH 6.8. Extrudates of HPC Klucel G or HPC ester and nifedipine (70:30) were investigated; the speed of the piston of the capillary rheometer for producing them is 0.28 mm/s, preheating to a temperature of 185° C. The measured values are each averages of at least two measurements. The absorption was measured at 340 nm and the active ingredient content was determined using a calibration line.

| | | Matrix: | Matrix: | Matrix: | Matrix: |
|---|---|---|---|---|---|
| | Matrix: | HPC | HPC | HPC | HPC |
| Time | Klucel | lactate | lactate | glycolide | glycolide |
| [h] | G | (0.40; 06) | (0.79; 04) | (0.53; 23H) | (0.95; 22H) |
| 0 | −0.6 | −0.6 | −0.6 | −0.6 | −0.6 |
| 1 | 14 | 18 | 10 | 17 | 17 |
| 2 | 24 | 33 | 16 | 34 | 29 |
| 3 | 33 | 51 | 21 | 51 | 41 |
| 4 | 41 | 67 | 26 | 65 | 52 |
| 5 | 50 | 80 | 31 | 74 | 62 |
| 6 | 62 | 83 | 36 | 77 | 72 |
| 7 | 73 | 83 | 42 | 78 | 78 |
| 8 | 79 | 83 | 49 | 79 | 80 |
| 9 | 82 | 83 | 55 | 79 | 82 |
| 10 | 84 | 82 | 59 | 79 | 82 |
| 11 | 85 | 81 | 61 | 79 | 83 |
| 12 | 85 | 80 | 62 | 78 | 83 |

The measured value of −0.6 in line 0 derives from a measurement error caused by the apparatus; the true value must, to be correct, be 0.

### RELEASE CHARACTERISTICS OF EXTRUDATES WITH NIMODIPINE AS ACTIVE INGREDIENT

The releases as a % of the single dose of 30 mg of active ingredient were measured by the EP/DAB paddle method with a stirrer speed of 50 rpm. The release medium was a buffer of pH 6.8 with 0.15% by weight sodium lauryl sulphate. Extrudates of HPC ester and nimodipine (70:30) were investigated; the speed of the piston of the capillary rheometer for producing them is 0.28 mm/s, preheating to a temperature of 145° C. The measured values are each averages of at least two measurements. The absorption was measured at 360 nm and the active ingredient content was determined using a calibration line.

| | Matrix: | Matrix: | Matrix: | Matrix: |
|---|---|---|---|---|
| Time | HPC lactate | HPC lactate | HPC glycolide | HPC glycolide |
| [h] | (0.79; 04) | (0.40; 06) | (0.95; 22H) | (0.53; 23H) |
| 0 | 0 | 0 | 0 | 0 |
| 1 | 17 | 26 | 19 | 13 |
| 2 | 40 | 56 | 40 | 33 |
| 3 | 56 | 75 | 57 | 51 |
| 4 | 66 | 84 | 70 | 65 |
| 5 | 74 | 86 | 80 | 74 |
| 6 | 78 | 86 | 86 | 78 |
| 7 | 81 | 87 | 91 | 80 |
| 8 | 82 | 88 | 94 | 81 |
| 9 | 82 | 88 | 96 | 81 |
| 10 | 83 | 88 | 97 | 81 |
| 11 | 83 | 88 | 97 | 82 |
| 12 | 83 | 88 | 98 | 82 |

1. Thermoplastic, water-soluble compounds of the general formula (I) 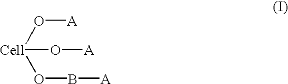
in which
Cell is the substituted residue of a hydroxyl group on the cellulose chain,
A is hydrogen or a hydroxycarboxylic acid residue,
B is an ether residue (—E—O)ₙ,
in which
n is 1 to 4, the molar degree of substitution by hydroxycarboxylic acid being between 0 and 1 and the molar degree of substitution of the ether being greater than or equal to 3,
E represents C₁-C₆-alkyl.

2. Cellulose derivatives according to claim 1, in which the ether is a propyl ether.

3. Cellulose derivatives according to claim 1 or 2, in which the hydroxycarboxylic acid is an α-hydroxycarboxylic acid.

4. Cellulose derivatives according to claim 3, in which the α-hydroxycarboxylic acid is lactic acid or hydroxyacetic acid.

5. Cellulose derivatives according to any of claims 1 to 4, characterized in that the molar degree of substitution by ether is 3 to 4.5.

6. Cellulose derivatives according to claim 5, characterized in that the molar degree of substitution by ether is 3.5 to 4.

7. Process for preparing cellulose derivatives according to claim 1, characterized in that the hydroxypropylcellulose is reacted with esters of the hydroxycarboxylic acid.

8. Process for preparing cellulose derivatives according to claim 1, characterized in that the hydroxypropylcellulose is reacted with dilactide or 1,4-dioxane-2,5-dione.

9. Use of the cellulose derivatives according to claim 1 for producing pharmaceutical preparations.

10. Pharmaceutical preparations comprising cellulose derivatives according to claim 1.

11. Use according to claim 9, where the pharmaceutical composition is a composition with modified release of active ingredient.

12. Use according to claim 11, where the pharmaceutical composition is a slow-release composition.
